(19) United States
(12) Patent Application Publication
(10) Pub. No.: US 2026/0199654 A1
(43) Pub. Date: Jul. 16, 2026

(54) Connector Assembly

(71) Applicant: Becton, Dickinson and Company, Franklin Lakes, NJ (US)

(72) Inventor: Zachary Jarrod Traina, Denville, NJ (US)

(21) Appl. No.: 19/136,369

(22) PCT Filed: Dec. 18, 2023
(86) PCT No.: PCT/US2023/084648

§ 371 Date: Jun. 6, 2025

Related U.S. Application Data
(60) Provisional application No. 63/433,611, filed on Dec. 19, 2022.

(51) Int. Cl. A61M 39/10

(52) U.S. Cl. CPC A61M 39/10; A61M 2039/1027; A61M 2039/1038; A61M 2039/1072; A61M 2205/0233; A61M 2205/18; A61M 2205/3327; A61M 2205/3592; A61M 2205/52

(57) ABSTRACT

A connector assembly may include an inner housing, a printed circuit board, an adhesive layer, a sensor film, and/or an outer housing. The inner housing may include a fluid flow path extending between a distal end and a proximal end of the inner housing. The printed circuit board may be on the inner housing. The printed circuit board may include a plurality of electrical contacts. The adhesive layer may be on the printed circuit board. The sensor film may be on the adhesive layer. The sensor film may be configured to generate a voltage in response to a force applied to the sensor film. The outer housing may surround the inner housing, the printed circuit board, the adhesive layer, and the sensor film.

### CROSS-REFERENCE TO RELATED APPLICATION

The present application claims priority to U.S. Provisional Patent Application No. 63/433,611 entitled “Connector Assembly” filed Dec. 19, 2022, the entire disclosure of which is hereby incorporated by reference in its entirety.

### BACKGROUND

A needleless connector may be scrubbed with a disinfectant to disinfect the needleless connector.

Further, identification of disposable medical devices is commonly performed with radio-frequency identification (RFID) tagging. Although robust and cost effective in higher volumes, RFID tagging uses significant operating power and increases cost, weight, and size of disposable medical devices, which may not be feasible for use with needleless connectors. RFID may also place limits on geometry and an orientation of a disposable medical device relative to a sensing device, which again, may not be feasible for use with needleless connectors.

### SUMMARY

According to some non-limiting embodiments or aspects, provided is a connector assembly, including: an inner housing including a fluid flow path extending between a distal end and a proximal end of the inner housing; a printed circuit board on the inner housing, the printed circuit board including a plurality of electrical contacts; an adhesive layer on the printed circuit board; a sensor film on the adhesive layer, the sensor film being configured to generate a voltage in response to a force applied to the sensor film; and an outer housing surrounding the inner housing, the printed circuit board, the adhesive layer, and the sensor film.

In some non-limiting embodiments or aspects, the printed circuit board surrounds the inner housing including the fluid flow path.

In some non-limiting embodiments or aspects, the plurality of electrical contacts are spaced apart from each other radially around the inner housing including the fluid flow path.

In some non-limiting embodiments or aspects, the printed circuit board includes a flexible printed circuit board that is wrapped around the inner housing including the fluid flow path.

In some non-limiting embodiments or aspects, the printed circuit board includes a plurality of printed circuit boards, and the connector assembly further includes: a plurality of rigid plates between the inner housing and the plurality of printed circuit boards, the plurality of printed circuit boards being connected to each other by the sensor film.

In some non-limiting embodiments or aspects, the adhesive layer includes an anisotropic conductive adhesive.

In some non-limiting embodiments or aspects, the adhesive layer covers at least one electrical contact of the plurality of electrical contacts, and the adhesive layer includes at least one opening in which at least one other electrical contact of the plurality of electrical contacts is located without being covered by the adhesive layer.

In some non-limiting embodiments or aspects, the connector assembly further includes: at least one actuator extending through an opening in the outer housing, the at least one actuator, in response to being actuated, being configured to force the sensor film into direct contact with the at least one other electrical contact and applies the force to the sensor film to generate the voltage in the sensor film.

In some non-limiting embodiments or aspects, the connector assembly further includes: a seal surrounding the inner housing, the printed circuit board, the adhesive layer, and the sensor film, the seal including the at least one actuator; a distal port connected to the distal end of the inner housing, the distal port including a septum, the seal being connected to a distal end of the outer housing such that an inner surface of the outer housing is spaced apart from the inner housing, the printed circuit board, the adhesive layer, and the sensor film, and the proximal end of the inner housing including a proximal port extending through and spaced apart from the outer housing.

In some non-limiting embodiments or aspects, the sensor film includes a polyvinylidene fluoride (PVDF) film.

In some non-limiting embodiments or aspects, the sensor film includes a first electrically conductive layer on the adhesive layer, a PVDF layer on the first electrical conductive layer, and a second electrically conductive layer on the PVDF layer.

In some non-limiting embodiments or aspects, a portion of the second electrically conductive layer is located between the PVDF layer and the adhesive layer.

In some non-limiting embodiments or aspects, one or more electrical signals are generated at one or more electrical contacts of the plurality of electrical contacts in response to the voltage generated in the sensor film in response to the force applied to the sensor film.

In some non-limiting embodiments or aspects, the connector assembly further includes: wireless communication circuitry configured to wirelessly communicate signal data associated with the one or more electrical signals to an external computing device.

In some non-limiting embodiments or aspects, the connector assembly further includes: at least one processor connected to the plurality of electrical contacts, and the at least one processor is programmed and/or configured to: receive, from the one or more electrical contacts of the plurality of electrical contacts, the one or more electrical signals generated in response to the voltage generated in the sensor film in response to the force applied to the sensor film; and determine, based on the one or more electrical signals, at least one event associated with the connector assembly, the signal data including the at least one event.

In some non-limiting embodiments or aspects, the at least one processor is further programmed and/or configured to determine the at least one event based on at least one of the following: an order in which the one or more electrical signals are received, one or more durations of the one or more electrical signals, or any combination thereof.

In some non-limiting embodiments or aspects, the at least one event includes a connection of a medical device to the connector assembly, and the at least one processor is further programmed and/or configured to determine, based on the one or more electrical signals, a type of the medical device from a plurality of different types of medical devices.

In some non-limiting embodiments or aspects, the at least one event includes a scrubbing event in which the connector assembly is disinfected.

According to some non-limiting embodiments or aspects, provided is a system, including: a connector assembly, including: an inner housing including a fluid flow path extending between a distal end and a proximal end; a printed circuit board on the inner housing, the printed circuit board including a plurality of electrical contacts; an adhesive layer on the printed circuit board; a sensor film on the adhesive layer, the sensor film being configured to generate a voltage in response to a force applied to the sensor film, and one or more electrical signals are generated at one or more electrical contacts of the plurality of electrical contacts in response to the voltage generated in the sensor film in response to the force applied to the sensor film; an outer housing surrounding the inner housing, the printed circuit board, the adhesive layer, and the sensor film; wireless communication circuitry configured to wirelessly communicate signal data associated with the one or more electrical signals to an external computing device; and at least one processor programmed and/or configured to: receive the signal data; determine, based on the signal data, at least one event associated with the connector assembly; and at least one of (i) store, in a database, the at least one event in association with a patient and/or a provider and (ii) provide, via an output component, an alert associated with the at least one event.

In some non-limiting embodiments or aspects, the connector assembly further includes a distal port connected to the distal end of the inner housing, and the system further includes: a medical device including: a barrel connection configured for complementary mating with the distal port; and an extension surrounding and spaced apart from the barrel connection, an inner surface of the extension that faces an outer surface of the barrel connection including at least one rib extending radially inward toward the barrel connection, and, in response to mating the barrel connection of the medical device with the distal port of the connector assembly, the at least one rib applies the force to the sensor film to generate the voltage in the sensor film.

Further non-limiting embodiments or aspects are set forth in the following numbered clauses:

Clause 1. A connector assembly, comprising: an inner housing including a fluid flow path extending between a distal end and a proximal end of the inner housing; a printed circuit board on the inner housing, wherein the printed circuit board includes a plurality of electrical contacts; an adhesive layer on the printed circuit board; a sensor film on the adhesive layer, wherein the sensor film is configured to generate a voltage in response to a force applied to the sensor film; and an outer housing surrounding the inner housing, the printed circuit board, the adhesive layer, and the sensor film.

Clause 2. The connector assembly of clause 1, wherein the printed circuit board surrounds the inner housing including the fluid flow path.

Clause 3. The connector assembly of any of clauses 1 and 2, wherein the plurality of electrical contacts are spaced apart from each other radially around the inner housing including the fluid flow path.

Clause 4. The connector assembly of any of clauses 1-3, wherein the printed circuit board includes a flexible printed circuit board that is wrapped around the inner housing including the fluid flow path.

Clause 5. The connector assembly of any of clauses 1-4, wherein the printed circuit board includes a plurality of printed circuit boards, and wherein the connector assembly further comprises: a plurality of rigid plates between the inner housing and the plurality of printed circuit boards, wherein the plurality of printed circuit boards is connected to each other by the sensor film.

Clause 6. The connector assembly of any of clauses 1-5, wherein the adhesive layer includes an anisotropic conductive adhesive.

Clause 7. The connector assembly of any of clauses 1-6, wherein the adhesive layer covers at least one electrical contact of the plurality of electrical contacts, and wherein the adhesive layer includes at least one opening in which at least one other electrical contact of the plurality of electrical contacts is located without being covered by the adhesive layer.

Clause 8. The connector assembly of any of clauses 1-7, further comprising: at least one actuator extending through an opening in the outer housing, wherein the at least one actuator, in response to being actuated, forces the sensor film into direct contact with the at least one other electrical contact and applies the force to the sensor film to generate the voltage in the sensor film.

Clause 9. The connector assembly of any of clauses 1-8, further comprising: a seal surrounding the inner housing, the printed circuit board, the adhesive layer, and the sensor film, wherein the seal includes the at least one actuator; a distal port connected to the distal end of the inner housing, wherein the distal port includes a septum, wherein the seal is connected to a distal end of the outer housing such that an inner surface of the outer housing is spaced apart from the inner housing, the printed circuit board, the adhesive layer, and the sensor film, and wherein the proximal end of the inner housing includes a proximal port extending through and spaced apart from the outer housing.

Clause 10. The connector assembly of any of clauses 1-9, wherein the sensor film includes a polyvinylidene fluoride (PVDF) film.

Clause 11. The connector assembly of any of clauses 1-10, wherein the sensor film includes a first electrically conductive layer on the adhesive layer, a PVDF layer on the first electrical conductive layer, and a second electrically conductive layer on the PVDF layer.

Clause 12. The connector assembly of any of clauses 1-11, wherein a portion of the second electrically conductive layer is located between the PVDF layer and the adhesive layer.

Clause 13. The connector assembly of any of clauses 1-12, wherein one or more electrical signals are generated at one or more electrical contacts of the plurality of electrical contacts in response to the voltage generated in the sensor film in response to the force applied to the sensor film.

Clause 14. The connector assembly of any of clauses 1-13, further comprising: wireless communication circuitry configured to wirelessly communicate signal data associated with the one or more electrical signals to an external computing device.

Clause 15. The connector assembly of any of clauses 1-14, further comprising: at least one processor connected to the plurality of electrical contacts, wherein the at least one processor is programmed and/or configured to: receive, from the one or more electrical contacts of the plurality of electrical contacts, the one or more electrical signals generated in response to the voltage generated in the sensor film in response to the force applied to the sensor film; and determine, based on the one or more electrical signals, at least one event associated with the connector assembly, wherein the signal data includes the at least one event.

Clause 16. The connector assembly of any of clauses 1-15, wherein the at least one processor is further programmed and/or configured to determine the at least one event based on at least one of the following: an order in which the one or more electrical signals are received, one or more durations of the one or more electrical signals, or any combination thereof.

Clause 17. The connector assembly of any of clauses 1-16, wherein the at least one event includes a connection of a medical device to the connector assembly, and wherein the at least one processor is further programmed and/or configured to determine, based on the one or more electrical signals, a type of the medical device from a plurality of different types of medical devices.

Clause 18. The connector assembly of any of clauses 1-17, wherein the at least one event includes a scrubbing event in which the connector assembly is disinfected.

Clause 19. A system, comprising: a connector assembly, including: an inner housing including a fluid flow path extending between a distal end and a proximal end; a printed circuit board on the inner housing, wherein the printed circuit board includes a plurality of electrical contacts; an adhesive layer on the printed circuit board; a sensor film on the adhesive layer, wherein the sensor film is configured to generate a voltage in response to a force applied to the sensor film, and wherein one or more electrical signals are generated at one or more electrical contacts of the plurality of electrical contacts in response to the voltage generated in the sensor film in response to the force applied to the sensor film; an outer housing surrounding the inner housing, the printed circuit board, the adhesive layer, and the sensor film; wireless communication circuitry configured to wirelessly communicate signal data associated with the one or more electrical signals to an external computing device; and at least one processor programmed and/or configured to: receive the signal data; determine, based on the signal data, at least one event associated with the connector assembly; and at least one of (i) store, in a database, the at least one event in association with a patient and/or a provider and (ii) provide, via an output component, an alert associated with the at least one event.

Clause 20. The system of clause 19, wherein the connector assembly further includes a distal port connected to the distal end of the inner housing, and wherein the system further comprises: a medical device including: a barrel connection configured for complementary mating with the distal port; and an extension surrounding and spaced apart from the barrel connection, wherein an inner surface of the extension that faces an outer surface of the barrel connection includes at least one rib extending radially inward toward the barrel connection, and wherein, in response to mating the barrel connection of the medical device with the distal port of the connector assembly, the at least one rib applies the force to the sensor film to generate the voltage in the sensor film.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIG. 1 is a perspective view of non-limiting embodiments or aspects of a connector assembly;

FIG. 2 is an exploded perspective view of non-limiting embodiments or aspects of a connector assembly;

FIG. 3 is a perspective view of non-limiting embodiments or aspects of components of a connector assembly;

FIG. 4 is a cross-sectional view of non-limiting embodiments or aspects of components of a connector assembly;

FIG. 5 is a perspective view of non-limiting embodiments or aspects of a system including a connector assembly; and

FIGS. 6A and 6B are perspective views of non-limiting embodiments or aspects of components of a medical device.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

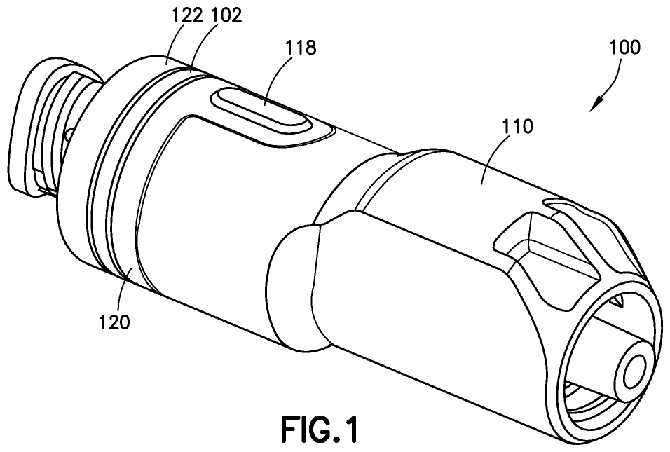

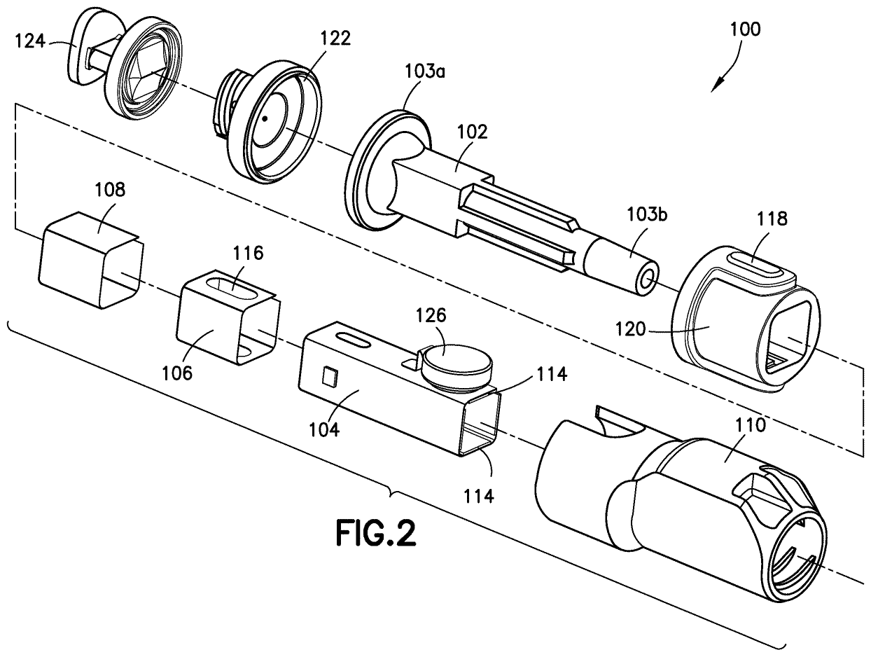

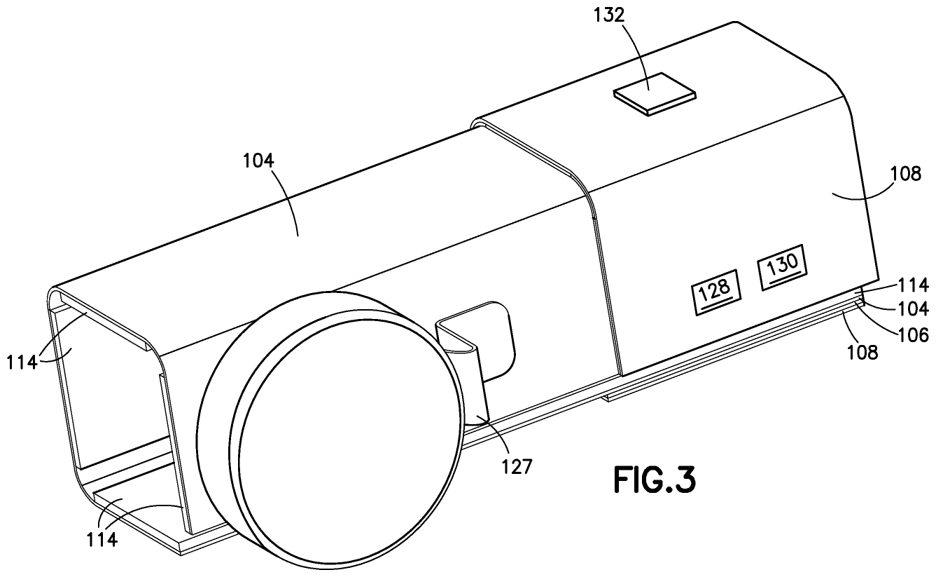

Referring now to FIGS. 1-6B, connector assembly 100 may include inner housing 102, printed circuit board 104, adhesive layer 106, sensor film 108 (composed of a first electrically conductive layer 109a, second electrically conductive layer 109b, and PVDF layer 111), and/or outer housing 110.

Inner housing 102 may include a fluid flow path extending between a distal end 103a and a proximal end 103b of inner housing 102.

Printed circuit board 104 may be on inner housing 102 (e.g., in direct contact with inner housing 102, connected to inner housing 102, etc.). For example, printed circuit board 104 may surround inner housing 102 including the fluid flow path. Printed circuit board 104 may include a plurality of electrical contacts 112a, 112b, . . . 112n. The plurality of electrical contacts may be spaced apart from each other radially around inner housing 102 including the fluid flow path (and/or in an axial direction of the fluid flow path).

In some non-limiting embodiments or aspects, printed circuit board 104 includes a flexible printed circuit board that is wrapped around inner housing 102 including the fluid flow path. In such an example, connector assembly 100 may include a plurality of rigid plates 114 between inner housing 102 and the flexible printed circuit board.

In some non-limiting embodiments or aspects, printed circuit board 104 includes a plurality of printed circuit boards. For example, the plurality of printed circuit boards may be spaced apart from each other around inner housing 102 including the fluid flow path. In such an example, connector assembly 100 may include a plurality of rigid plates 114 between inner housing 102 and the plurality of printed circuit boards, and/or the plurality of printed circuit boards may include a plurality of rigid printed circuit boards. In such an example, the plurality of printed circuit boards may connected to each other by sensor film 108.

Adhesive layer 106 may be on printed circuit board 104 (e.g., in direct contact with printed circuit board 104, connected to printed circuit board 104, etc.). For example, adhesive layer 106 may surround inner housing 102 including the fluid flow path and/or printed circuit board 104. Adhesive layer 106 may include an anisotropic conductive adhesive (e.g., 3M™ Electrically Conductive Adhesive Transfer Tape 9703, etc.). For example, adhesive layer 106 may include a z-axis anisotropic conductive adhesive configured to be electrically conductive only through a thickness of adhesive layer 106 (e.g., vertically between printed circuit board 104 and sensor film 108, but not horizontally across adhesive layer 106, etc.).

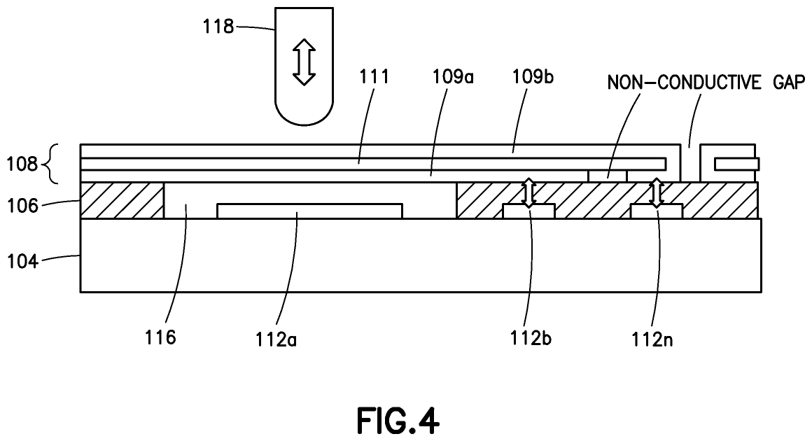

Adhesive layer 106 may cover at least one electrical contact of the plurality of electrical contacts 112a, 112b, . . . 112n. For example, as shown in FIG. 4, adhesive layer 106 may cover electrical contacts 112b and 112n. Adhesive layer 106 may include at least one opening in which at least one other electrical contact of the plurality of electrical contacts 112a, 112b, . . . 112n is located without being covered by the adhesive layer. For example, as also shown in FIG. 4, adhesive layer 106 may include opening 116 in which electrical contact 112a is located without being covered by the adhesive layer. As an example, opening 116 may include an air gap between electrical contact 112a and sensor film 108, and/or the anisotropic conductive adhesive of adhesive layer 106 may be between electrical contacts 112b and 112n and sensor film 108. While not shown in FIG. 4, it is recognized that in some embodiments, the electrical contact 112a may be split further into multiple contacts, with corresponding breaks or gaps provided in the first electrically conductive 109a, such that a membrane switch circuit formed by sensor film 108 and electrical contact 112a is electrically isolated/distinct from a piezoelectric circuit formed by sensor film 108 and electrical contact 112b.

In some non-limiting embodiments or aspects, connector assembly 100 further includes at least one actuator extending through an opening in outer housing 110. For example, as shown in FIG. 1, actuator 118 (e.g., a depressible button, etc.) may extend through an opening in housing 110. As an example, actuator 118, in response to being actuated, may force sensor film 108 into direct contact with electrical contact 112a and/or apply the force to sensor film 108 to generate a voltage in sensor film 108.

In some non-limiting embodiments or aspects, connector assembly 100 further includes seal 120 and/or distal port 122. Seal 120 (e.g., an elastomeric seal, etc.) may surround inner housing 102, printed circuit board 104, adhesive layer 106, and/or sensor film 108, and/or seal 120 may include actuator 118. Distal port 122 may be connected to the distal end 103a of inner housing 102. Distal port 122 may include a luer-lock connection. Distal port may include septum 124. For example, distal port 122 and septum 124 may form a valve that is received in and/or integrated with the distal end 103a of inner housing 102 (e.g., of a valve retainer, etc.).

Battery 126 may be connected to printed circuit board 104 (e.g., via battery contact 127, etc.) to supply power to sensor film 108 of for generating the voltage in sensor film 108 (e.g., between first electrically conductive layer 109a and second electrically conductive layer 109b and/or one or more electrical contacts 112, etc.) in response to a force applied to sensor film 108. Battery 126 may include a rechargeable battery, a single use battery, a replaceable battery, or any combination thereof. In some non-limiting embodiments or aspects, battery 126 includes an energy harvester (e.g., a thermoelectric energy harvester, a photovoltaic energy harvester, a piezoelectric energy harvester, etc.).

Printed circuit board 104 may further include a processor 128 (e.g., a low power microcontroller unit (MCU), etc.), wireless communication circuitry 130, and/or a user input/feedback device 132 (e.g., a light emitting diode (LED) and/or switch, etc.), and battery 126 may be configured to supply power to these and/or any other electrical components of connector assembly 100. For example, printed circuit board 104 (and/or processor 128) may be electrically connected to and/or electrically interconnect sensor film 108, electrical contacts 112a, 112b, . . . 112n, battery 126, wireless communication circuitry 130, user input/feedback device 130.

User input/feedback device 132 may be configured to receive a user input from a user and/or to provide feedback to the user. For example, user input/feedback device 132 may include at least one of the following: a button/switch, a display, a light-emitting diode (LED), an audio output device (e.g., a buzzer, a speaker, etc.), or any combination thereof. User input/feedback device 132 may extend through an opening and/or be visible through outer housing 110 to be visible by and accessible to a user, and/or user input/feedback device 132 may be integral to outer housing 110 (e.g., overmolded, etc.). In some non-limiting embodiments or aspects, user input/feedback device 132 may include actuator 118.

Outer housing 110 may surround inner housing 102, printed circuit board 104, adhesive layer 106, and/or sensor film 108. Seal 120 may be connected to a distal end of outer housing 110 such that an inner surface of outer housing 110 (e.g., a surface that faces inner housing 102, etc.) is spaced apart from inner housing 102, printed circuit board 104, adhesive layer 106, and/or sensor film 108. The proximal end 103b of inner housing 102 may include a proximal port extending proximally though and spaced apart from outer housing 110. For example, outer housing 110 may be secured and/or gimballed with the distal end 103a of inner housing 102 to float relative to inner housing 102, printed circuit board 104, adhesive layer 106, and/or sensor film 108.

Sensor film 108 may be on adhesive layer 106 (e.g., in direct contact with adhesive layer 106, connected to adhesive layer 106, etc.). For example, sensor film 108 may surround inner housing 102 including the fluid flow path, printed circuit board 104, and/or adhesive layer 106. Sensor film 108 may be configured to generate a voltage in response to a force applied to sensor film 108.

In some non-limiting embodiments or aspects, sensor film 108 includes a polyvinylidene fluoride (PVDF) film. For example, and referring specifically to FIG. 4, sensor film 108 may include first electrically conductive layer 109a on adhesive layer 106, PVDF layer 111 on first electrical conductive layer 109a, and second electrically conductive layer 109b on PVDF layer 111. In such an example, a portion of second electrically conductive layer 109b may located between PVDF layer 111 and the adhesive layer 106. For example, second electrically conductive layer 109b may wrap around an end of PVDF layer 111 and sit directly between PVDF layer 111 and adhesive layer 106 at a location where first electrically conductive layer 109a does not extend between PVDF layer 111 and adhesive layer 106 (e.g., at a location corresponding to electrical contact 112n on printed circuit board 104.

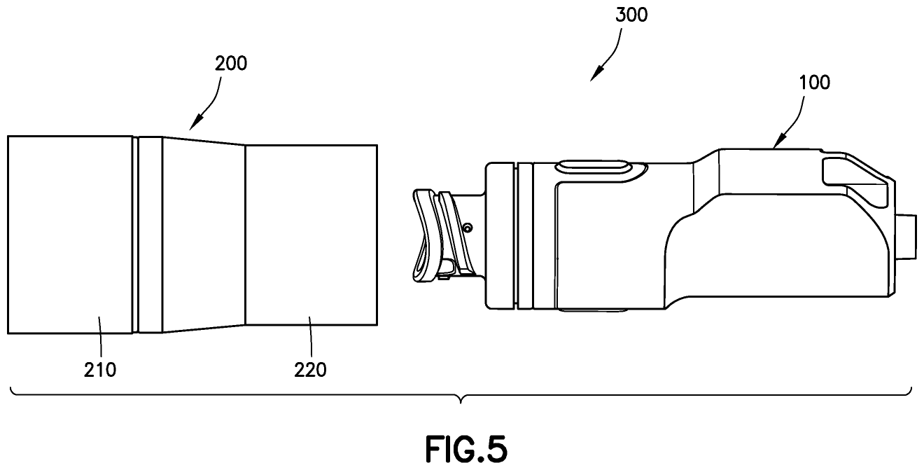

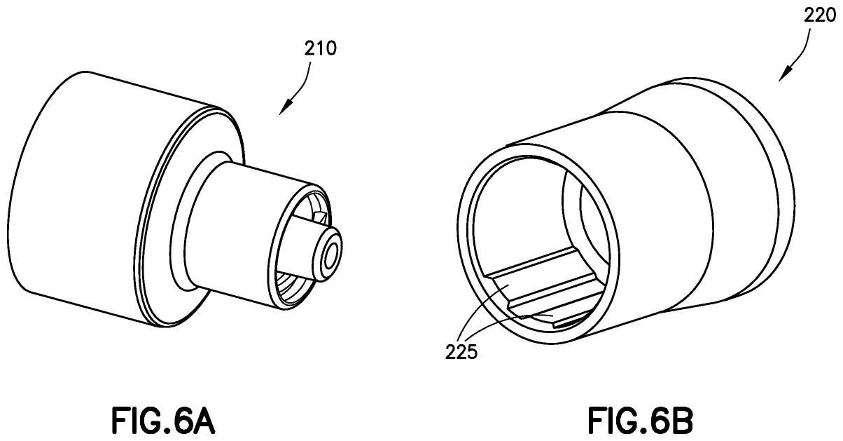

One or more electrical signals may be generated at one or more electrical contacts of the plurality of electrical contacts 112a, 112b, . . . 112n in response to the voltage generated in sensor film 106 in response to the force applied to sensor film 106. For example, a relative motion applied to connector assembly 100 (e.g., during scrubbing/disinfection, etc.) may pinch sensor film 108 to activate radial switches formed by the plurality of electrical contacts 112a, 112b, . . . 112n and sensor film 108 (e.g., for the dual purpose of receiving user input and/or device differentiation, etc.), and/or sensor film 108 may be configured to act simultaneously as a membrane switch in areas where static load sensing is desired (e.g., beneath actuator 118, etc.). As an example, different-shaped disposables or devices (e.g., medical device 200 described herein below in more detail with respect to FIGS. 5, 6A, and 6B, etc.) may selectively activate the radial switches to identify connection of the disposables and/or types of the disposables. For example, devices can be differentiated in their final position (e.g., at a 2ⁿ device resolution, where n=a number of the switches formed by the plurality of electrical contacts 112a, 112b, . . . 112n and sensor film 108). In such an example, the device differentiation switches may be operated concurrently and/or in time domain to provide more than a 2ⁿ device resolution. As an example, sensor film 108 may be sub-divided into multiple sensor elements corresponding, for example, to a scrubbing event, a device detection connection event, and/or the like. As an example, sensor film 108 and electrical contact 112a may operate as a membrane switch beneath actuator 118 to detect a static load (e.g. to detect a device type/presence, etc.) and/or sensor film 108 and electrical contacts 112b and 112n may operate to sense dynamic loads (e.g., from scrubbing/disinfecting that applies a “pinching” pressure to sensor film 108) on connector assembly 100.

For example, and referring again to FIG. 4, adhesive layer 106 may electrically connect second electrically conductive layer 109b and electrical contact 112n and/or electrically connect first electrically conductive layer 109a and electrical contact 112 to form dynamic switches, and/or actuator 118 may deflect sensor film 108 to bring second electrically conductive layer 109a into contact electrical switch 112a to form a static switch, with dynamic loads imparted on sensor film 108 (in either areas supported or unsupported by adhesive layer 106) generating the voltage response between first electrically conductive layer 109a and second electrically conductive layer 109b of sensor film 108. As an example, periodic/intermittent loads on connector assembly 100 from scrubbing/disinfection may generate a corresponding periodic/intermittent voltage in sensor film 108 (e.g., due to piezoelectric properties of sensor film 108, etc.), and by nature of the film and the corresponding driving electronic components, this generated voltage may be transient (e.g., the voltage may persist only as long as the input load is varying, etc.). As an example, the same sensor film 108 may be spaced apart from a mating contact by adhesive layer 106 (e.g., a perforated, pressure sensitive adhesive, where sensor film 108 itself may be employed as one half of a membrane switch, etc.), and applying a load (e.g., via actuator 118, etc.) may deflect sensor film 108 into contact with electrical contact 112a, completing a contact circuit. In such an example, a geometry and thickness of the spacer may be configured such that contact is initiated/terminated at a designed load.

In this way, sensor film 108 and electrical contact 112a, 112b, . . . 112n may act as static and dynamic sensors, for example as part of a “smart” needleless connector, where it may be desirable to sense both dynamic inputs (e.g., scrubbing or manual disinfection, etc.) and static inputs (e.g., a presence or absence of attached devices, etc.), and/or sensor film 108 and adhesive layer 106 may provide a desired or arbitrary number of collocated state and/or dynamic switches, with minimal impact on manufacturing complexity and cost. For example, re-use of the same sensor in two modes may have an advantage of reducing both size and cost and/or may be a significant architectural simplification as compared to alternate approaches that may employ separate sensors and/or different sensing modalities.

Wireless communication circuitry 130 may be configured to wirelessly communicate (e.g., via a short range wireless communication connection, such as an NFC communication connection, an RFID communication connection, a Bluetooth® communication connection, and/or the like, etc.) information and/or data with an external computing device. Wireless communication circuitry 130 may be configured to establish communication with an external computing device (e.g., pair and/or activate a pairing sequence for pairing with the external computing device, etc.) based on a predetermined user input to the user input/feedback device 132 and/or actuator 118 (e.g., in response to a user pressing and/or holding a button, etc.). Wireless communication circuitry 130 may be configured to wirelessly communicate, to an external computing device, signal data associated with the one or more electrical signals generated at the one or more electrical contacts of the plurality of electrical contacts 112a, 112b, . . . 112n in response to the voltage generated in sensor film 108 in response to the force applied to the sensor film.

Processor 128 may be connected to the plurality of electrical contacts 112a, 112b, . . . 112n. For example, processor 128 may be programmed and/or configured to receive, from the one or more electrical contacts of the plurality of electrical contacts 112a, 112b, . . . 112n, the one or more electrical signals generated in response to the voltage generated in sensor film 108 in response to the force applied to sensor film 108. As an example, processor 128 may determine, based on the one or more electrical signals, at least one event associated with the connector assembly, and/or the signal data transmitted by wireless communication circuitry 130 may include the at least one event. In such an example, processor 128 may be programmed and/or configured to determine the at least one event based on at least one of the following: an order in which the one or more electrical signals are received, one or more durations of the one or more electrical signals, or any combination thereof. The determination of the least one event by processor 128 may be performed via a comparison of the received one or more electrical signals to a plurality of electrical signal patterns stored in a reference database—with the processor 128 matching the received one or more electrical signals to an electrical signal pattern stored in the database to identify the type of event experienced by the connector assembly 100. For example, as described herein above, sensor film 108 and adhesive layer 106 may provide a desired or arbitrary number of collocated state and/or dynamic switches that may be read by processor 128 to determine the at least one event. As an example, the at least one event may include a connection of medical device 200 to the connector assembly, and/or processor 128 may be programmed and/or configured to determine, based on the one or more electrical signals, a type of medical device 200 from a plurality of different types of medical devices. As an example, the at least one event may include a scrubbing event in which connector assembly 100 is disinfected.

In one example, processor 128 may identify that the connector assembly 100 is being exposed to a scrubbing event via which the connector assembly 100 is disinfected. The processor 128 may receive a number of electrical signals from the electrical contacts 112a, 112b, . . . 112n responsive to a force applied to the sensor film 108 from the scrubbing/disinfection—with the electrical signals provided as periodic/intermittent signals (i.e., transient signals). The processor 128 may compare the pattern of the periodic/intermittently received signals (i.e., an order in which the electrical signals are received and/or durations of the electrical signals) to a plurality of electrical patterns stored in a database (e.g., a database stored in a memory associated with the processor and/or a database associated with an external computing system, etc.), and match the pattern to a stored electrical pattern indicating that the pattern is associated with a scrubbing/disinfecting event.

In another example, processor 128 may identify that the connector assembly 100 is being connected to a medical device 200. The processor 128 may receive one or electrical signals from the electrical contacts 112a, 112b, . . . 112n responsive to a force applied to the sensor film 108 from the connection of medical device 200—with the electrical signals provided as static signals. The processor 128 may compare the pattern of the received signals (i.e., an order in which the electrical signals are received and/or durations of the electrical signals) to a plurality of electrical patterns stored in a database (e.g., a database stored in a memory associated with the processor and/or a database associated with an external computing system, etc.), and match the pattern to a stored electrical pattern indicating that the pattern is associated with connection of a medical device 200. In some embodiments, each medical device 200 may provide a unique timing of switch presses and/or of multiple concurrent switch presses when connected to connector assembly 100 that generates a unique electrical signal pattern that may be read by the processor 128 (and compared to stored electrical signal patterns), to enable identification of the specific type of medical device (from a plurality of different types of medical devices) that is connected to the connector assembly 100.

In some non-limiting embodiments or aspects, and referring specifically to FIGS. 5, 6A, and 6B a system 300 may include connector assembly 100, medical device 200, and/or an external computing device (not shown). For example, processor 128 may be located in connector assembly 100 or in the external computing device, and/or processor 128 may be implemented as multiple processors distributed across connector assembly 100 and/or the external computing device. In such an example, processor 128 may be programmed and/or configured to at least one of (i) store, in a database (e.g., a database associated with the external computing system, etc.), the at least one event in association with a patient, a provider, and/or a procedure and (ii) provide, via an output component (e.g., via user feedback device 132, via a display associated with the external computing system, etc.), an alert associated with the at least one event.

Medical device 200 may include barrel connection 210 configured for complementary mating with distal port 122 of connector assembly 100 and/or extension 220 surrounding and spaced apart from barrel connection 210. An inner surface of extension 220 that faces an outer surface of barrel connection 210 may include at least one rib 225 extending radially inward toward barrel connection 210. For example, in response to mating barrel connection 210 of medical device 200 with distal port 122 of connection assembly 100, the at least one rib 225 may apply the force to sensor film 108 to generate the voltage in sensor film 108. In this way, different configurations of the at least one rib 225 may be used to generate different timing of switch presses and/or multiple switch presses that may be read as unique patterns or codes for each different medical device or disposable when connected to connector assembly 100.

For example, the at least one rib 225 of medical device 200 may be configured to activate two or more switches in an alternating pattern, or a single button may be used as a timing track to signal when other switches should be poled for their state (e.g., similar to an I2C approach, etc.). Additional, or alternate, schemes may be used depending on a number of medical devices 200 or disposables to be differentiated and/or on whether the code is robust to partial assembly or intermittent screwing/unscrewing of the disposable, which noise sources may exist in the environment, etc.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A connector assembly, comprising:
an inner housing including a fluid flow path extending between a distal end and a proximal end of the inner housing;
a printed circuit board on the inner housing, wherein the printed circuit board includes a plurality of electrical contacts;
an adhesive layer on the printed circuit board;
a sensor film on the adhesive layer, wherein the sensor film is configured to generate a voltage in response to a force applied to the sensor film; and
an outer housing surrounding the inner housing, the printed circuit board, the adhesive layer, and the sensor film.

2. The connector assembly of claim 1, wherein the printed circuit board surrounds the inner housing including the fluid flow path.

3. The connector assembly of claim 2, wherein the plurality of electrical contacts are spaced apart from each other radially around the inner housing including the fluid flow path.

4. The connector assembly of claim 2, wherein the printed circuit board includes a flexible printed circuit board that is wrapped around the inner housing including the fluid flow path.

5. The connector assembly of claim 2, wherein the printed circuit board includes a plurality of printed circuit boards, and wherein the connector assembly further comprises:
a plurality of rigid plates between the inner housing and the plurality of printed circuit boards, wherein the plurality of printed circuit boards is connected to each other by the sensor film.

6. The connector assembly of claim 1, wherein the adhesive layer includes an anisotropic conductive adhesive.

7. The connector assembly of claim 6, wherein the adhesive layer covers at least one electrical contact of the plurality of electrical contacts, and wherein the adhesive layer includes at least one opening in which at least one other electrical contact of the plurality of electrical contacts is located without being covered by the adhesive layer.

8. The connector assembly of claim 1, further comprising:
at least one actuator extending through an opening in the outer housing, wherein the at least one actuator, in response to being actuated, forces the sensor film into direct contact with the at least one other electrical contact and applies the force to the sensor film to generate the voltage in the sensor film.

9. The connector assembly of claim 8, further comprising:
a seal surrounding the inner housing, the printed circuit board, the adhesive layer, and the sensor film, wherein the seal includes the at least one actuator;
a distal port connected to the distal end of the inner housing, wherein the distal port includes a septum,
wherein the seal is connected to a distal end of the outer housing such that an inner surface of the outer housing is spaced apart from the inner housing, the printed circuit board, the adhesive layer, and the sensor film, and wherein the proximal end of the inner housing includes a proximal port extending through and spaced apart from the outer housing.

10. The connector assembly of claim 1, wherein the sensor film includes a polyvinylidene fluoride (PVDF) film.

11. The connector assembly of claim 10, wherein the sensor film includes a first electrically conductive layer on the adhesive layer, a PVDF layer on the first electrical conductive layer, and a second electrically conductive layer on the PVDF layer.

12. The connector assembly of claim 11, wherein a portion of the second electrically conductive layer is located between the PVDF layer and the adhesive layer.

13. The connector assembly of claim 1, wherein one or more electrical signals are generated at one or more electrical contacts of the plurality of electrical contacts in response to the voltage generated in the sensor film in response to the force applied to the sensor film.

14. The connector assembly of claim 13, further comprising:
wireless communication circuitry configured to wirelessly communicate signal data associated with the one or more electrical signals to an external computing device.

15. The connector assembly of claim 14, further comprising:
at least one processor connected to the plurality of electrical contacts, wherein the at least one processor is programmed and/or configured to:
receive, from the one or more electrical contacts of the plurality of electrical contacts, the one or more electrical signals generated in response to the voltage generated in the sensor film in response to the force applied to the sensor film; and
determine, based on the one or more electrical signals, at least one event associated with the connector assembly, wherein the signal data includes the at least one event.

16. The connector assembly of claim 15, wherein the at least one processor is further programmed and/or configured to determine the at least one event based on at least one of the following: an order in which the one or more electrical signals are received, one or more durations of the one or more electrical signals, or any combination thereof.

17. The connector assembly of claim 15, wherein the at least one event includes a connection of a medical device to the connector assembly, and wherein the at least one processor is further programmed and/or configured to determine, based on the one or more electrical signals, a type of the medical device from a plurality of different types of medical devices.

18. The connector assembly of claim 15, wherein the at least one event includes a scrubbing event in which the connector assembly is disinfected.

19. A system, comprising:
a connector assembly, including:
an inner housing including a fluid flow path extending between a distal end and a proximal end;
a printed circuit board on the inner housing, wherein the printed circuit board includes a plurality of electrical contacts;
an adhesive layer on the printed circuit board;
a sensor film on the adhesive layer, wherein the sensor film is configured to generate a voltage in response to a force applied to the sensor film, and wherein one or more electrical signals are generated at one or more electrical contacts of the plurality of electrical contacts in response to the voltage generated in the sensor film in response to the force applied to the sensor film;
an outer housing surrounding the inner housing, the printed circuit board, the adhesive layer, and the sensor film;
wireless communication circuitry configured to wirelessly communicate signal data associated with the one or more electrical signals to an external computing device; and
at least one processor programmed and/or configured to:
receive the signal data;
determine, based on the signal data, at least one event associated with the connector assembly; and
at least one of (i) store, in a database, the at least one event in association with a patient and/or a provider and (ii) provide, via an output component, an alert associated with the at least one event.

20. The system of claim 19, wherein the connector assembly further includes a distal port connected to the distal end of the inner housing, and wherein the system further comprises:
a medical device including:
a barrel connection configured for complementary mating with the distal port; and
an extension surrounding and spaced apart from the barrel connection,
wherein an inner surface of the extension that faces an outer surface of the barrel connection includes at least one rib extending radially inward toward the barrel connection, and wherein, in response to mating the barrel connection of the medical device with the distal port of the connector assembly, the at least one rib applies the force to the sensor film to generate the voltage in the sensor film.
